(19) United States
(12) Patent Application Publication
(10) Pub. No.: US 2009/0264784 A1
(43) Pub. Date: Oct. 22, 2009

(54) APPARATUS AND METHOD FOR CREATING MULTIPLE FILTERED OUTPUTS FROM A SINGLE SENSOR

(75) Inventor: Peter Stasz, Moundsview, MN (US)

Correspondence Address: SCHWEGMAN, LUNDBERG & WOESSNER, P.A., P.O. BOX 2938, MINNEAPOLIS, MN, 55402, US

(73) Assignee: Dymedix Corporation, Shoreview, MN (US)

(21) Appl. No.: 12/425,820

(22) Filed: Apr. 17, 2009

Related U.S. Application Data
(60) Provisional application No. 61/045,735, filed on Apr. 17, 2008.

(51) Int. Cl. A61B 5/08

(52) U.S. Cl. 600/529

(57) ABSTRACT

Multiple different output signals for a polysomnograph (PSG) machine, the multiple different output signals including a first output indicative of an upper airway restriction (UAR), a second output indicative of an airway pressure during respiration, and a third output indicative of an airway air temperature during respiration, can be produced using a single sensor input.

### CLAIM OF PRIORITY

This patent application claims the benefit of priority to U.S. Provisional Patent Application Ser. No. 61/045,735, filed on Apr. 17, 2008, which application is herein incorporated by reference in its entirety.

### TECHNICAL FIELD

The present subject matter relates generally to an electronic signal processing circuit for adapting a piezo/pyro electric sensor to a conventional polysomnograph (PSG) machine of the type commonly used in sleep laboratory applications, and more particularly to an adapter module that receives a single incoming sensor signal and creates multiple signal outputs with different waveform shapes based on selected filter cut-off frequencies.

### BACKGROUND

Sleep disorders have recently become the focus of a growing number of physicians. Many hospitals and clinics have established sleep laboratories (sleep labs) to diagnose and treat sleep disorders such as sleep apnea, insomnia, and other physiological events or conditions occurring during sleep. In the sleep laboratories, practitioners use instrumentation to monitor and record a patient's sleep patterns. Practitioners rely on these recorded sleep patterns to diagnose patients and prescribe proper therapies.

The instrumentation used to record the sleep patterns generally includes sensors attached to a patient and connected via electrical leads to a polysomnograph (PSG) machine, which produces a waveform for interpretation by a practitioner. Several varieties of these sensors have been developed and commonly function by converting a mechanical bodily movement to an electrical signal related to the body movement.

Air pressure transducers (APT) and thermistors (Thermo) represent the classical sensors used to record oral and nasal airflow. APT's are used in conjunction with a cannula attached to an air pressure hose. The APT cannula is placed under a patient's nose and measures differences in respiratory air pressure between inhaling and exhaling. Thermo sensors are placed under a patient's nose and measure differences in respiratory air temperature between inhaling and exhaling. As the classical sensors, both the APT and Thermo sensors produce a signal that presents a distinct and familiar waveform on the PSG machine. Unfortunately, due to their physical construction, chemical composition and solid state physics, neither of these sensors provide sufficient detail relating to upper airway restrictions (UAR). This makes it difficult or impossible for practitioners to recognize certain UAR events related to a patient's sleep disorder.

As an alternative to APT and Thermo sensors, Dymedix Corporation, applicant's assignee, recently introduced a new piezo/pyro sensor comprising a polyvinylidene (PVDF) film that is found to exhibit both piezoelectric and pyroelectric properties. Information regarding this type of sensor may be found in U.S. Pat. No. 5,311,875 to Stasz and U.S. Pat. No. 6,254,545 to Stasz et al. Piezo/pyro sensors of the type described may be adapted to be affixed to a subject's upper lip. In this condition, airflow in and out of the nostrils of a patient, due to inspiration and expiration, impinges on the sensor, which produces an output signal related to temperature and pressure changes occasioned by the inspiratory and expiratory flow. This sensor provides more detailed information regarding UAR's.

However, as a result of the more detailed information, one problem with this newly developed piezo/pyro sensor is that its signal produces a waveform on a PSG machine that is unfamiliar to sleep laboratory practitioners. Generally speaking, this is because the detailed information causes the waveform to differ from the distinct and familiar waveforms associated with the known APT and Thermo sensors discussed above.

There is added value in the detailed information produced by the piezo/pyro sensor and thus there is a need in the art for making its associated waveform familiar to sleep laboratory practitioners. Additionally, to successfully market these new types of sensors, it is desirable that they be able to be used with existing PSG machines already in place in sleep laboratories.

### SUMMARY

An adaptor module can be provided for interfacing a piezo/pyro electric film sensor to a PSG machine. In some embodiments, the adaptor module comprises a differential input amplifier having a pair of input terminals that are adapted to be coupled to the piezo/pyro electric film sensor and an output terminal. The differential input amplifier may be configured to significantly attenuate common-mode noise while providing a predetermined gain factor by which the sensor output signal is amplified. The output of the differential amplifier may be fed into a filter bank of multiple filter circuits.

In one embodiment the waveform of the piezo/pyro electric sensor output signal is shaped to resemble the waveform of an air pressure transducer and a thermistor that a diagnosing sleep disorder professional would see and recognize on a PSG.

In another embodiment, a differential input amplifier with a predetermined gain factor and appropriate conditioning of the amplified piezo/pyro sensor output signal allows three different filters to be readily matched to existing PSG electronic head boxes already on hand in most sleep laboratories.

In an example, multiple different output signals for a polysomnograph (PSG) machine, the multiple different output signals including a first output indicative of an upper airway restriction (UAR), a second output indicative of an airway pressure during respiration, and a third output indicative of an airway air temperature during respiration, can be produced using a single sensor input.

In Example 1, an apparatus for creating multiple filtered outputs for a polysomnograph (PSG) machine from a single sensor input includes an electronic signal processing circuit configured to receive a single sensor input and to produce, using the single sensor input, multiple different output signals, the multiple different output signals including a first output indicative of an upper airway restriction (UAR), a second output indicative of an airway pressure during respiration, and a third output indicative of an airway air temperature during respiration.

In Example 2, the second output of Example 1 is optionally indicative of a difference in the airway pressure during respiration, and the third output is indicative of a difference in the airway air temperature during respiration.

In Example 3, the electronic signal processing circuit of any one or more of Examples 1-2 is optionally configured to receive the single sensor input from a piezo/pyro sensor, sized and shaped to be affixed to a subject's upper lip and configured to receive respiration information from the subject.

In Example 4, the electronic signal processing circuit of any one or more of Examples 1-3 is optionally configured to provide information about at least one of the produced multiple different output signals to a user.

In Example 5, the electronic signal processing circuit of any one or more of Examples 1-4 is optionally configured to produce the multiple different output signals for a polysomnograph (PSG) machine from the single sensor input.

In Example 6, the electronic signal processing circuit of any one or more of Examples 1-5 optionally includes a differential amplifier configured to amplify the single sensor input and to attenuate common-mode noise.

In Example 7, the electronic signal processing circuit of any one or more of Examples 1-6 optionally includes a UAR shape filter configured to produce the first output, an air pressure transducer (APT) shape filter configured to produce the second output, and a thermistor (Thermo) shape filter configured to produce the third output.

In Example 8, the UAR shape filter of any one or more of Examples 1-7 optionally includes a first low-pass filter having a cut-off frequency between 1.5 Hz and 10 Hz, the APT shape filter of any one or more of Examples 1-7 optionally includes a second low-pass filter having a cut-off frequency between 0.5 Hz and 1.5 Hz, and the Thermo shape filter of any one or more of Examples 1-7 optionally includes a third low-pass filter having a cut-off frequency between 0.01 Hz and 0.5 Hz.

In Example 9, the electronic signal processing circuit of any one or more of Examples 1-8 is optionally configured to produce the second output to resemble an air pressure transducer (APT) waveform on a polysomnograph (PSG) machine, and to produce the third output to resemble a thermistor (Thermo) waveform on the PSG machine.

In Example 10, the electronic signal processing circuit of any one or more of Examples 1-9 is optionally configured to be integrated into a cable coupling a piezo/pyro sensor to a polysomnograph (PSG) machine.

In Example 11, a system for creating multiple filtered outputs for a polysomnograph (PSG) machine from a single sensor input includes a piezo/pyro sensor, sized and shaped to be affixed to a subject's upper lip, the piezo/pyro sensor configured to receive respiration information from the subject. The system also includes an electronic signal processing circuit configured to receive information from the piezo/pyro sensor and to produce, using the piezo/pyro sensor input, multiple different output signals, the multiple different output signals including a first output indicative of an upper airway restriction (UAR), a second output indicative of an airway pressure during respiration, and a third output indicative of an airway air temperature during respiration. Further, the system includes a polysomnograph (PSG) machine configured to receive the multiple different output signals from the electronic signal processing circuit and to display information about at least one of the received first output, the received second output, or the received third output to a user.

In Example 12, the system of Example 11 optionally includes a cable configured to couple the piezo/pyro sensor to the PSG machine, wherein the electronic signal processing circuit is configured to be integrated into the cable.

In Example 13, a method for creating multiple filtered outputs for a polysomnograph (PSG) machine from a single sensor input includes receiving a single sensor input, producing, using the single sensor input, multiple different output signals, the producing including producing a first output indicative of an upper airway restriction (UAR), producing a second output indicative of an airway pressure during respiration, and producing a third output indicative of an airway air temperature during respiration.

In Example 14, the receiving the single sensor input of Example 13 optionally includes receiving a single sensor input from a piezo/pyro sensor, sized and shaped to be affixed to a subject's upper lip and configured to receive respiration information from the subject.

In Example 15, the producing the multiple different output signals of any one or more of Examples 13-14 optionally includes using an electronic signal processing circuit integrated into a cable coupling the piezo/pyro sensor to a polysomnograph (PSG) machine.

In Example 16, the method of any one or more of Examples 13-15 optionally includes providing information about at least one of the produced multiple different output signals to a user.

In Example 17, the method of any one or more of Examples 13-16 optionally includes receiving the produced multiple different output signals using a polysomnograph (PSG) machine and providing information about at least one of the received multiple different output signals to a user.

In Example 18, the producing the first output of any one or more of Examples 13-17 optionally includes using a first UAR shape filter, the producing the second output includes using an air pressure transducer (APT) shape filter, and the producing the third output includes using a thermistor (Thermo) shape filter.

In Example 19, the using the UAR shape filter of any one or more of Examples 13-18 optionally includes using a first low-pass filter having a cut-off frequency between 1.5 Hz and 10 Hz, the using the APT shape filter of any one or more of Examples 13-18 optionally includes using a second low-pass filter having a cut-off frequency between 0.5 Hz and 1.5 Hz, and the using the Thermo shape filter of any one or more of Examples 13-18 optionally includes using a third low-pass filter having a cut-off frequency between 0.01 Hz and 0.5 Hz.

In Example 20, the producing the second output of any one or more of Examples 13-19 optionally includes producing output to resemble an air pressure transducer (APT) waveform on a polysomnograph (PSG) machine, and the producing the third output of any one or more of Examples 13-19 optionally includes producing output to resemble a thermistor (Thermo) waveform on the PSG machine.

Further areas of applicability will become apparent from the description provided herein. It should be understood that the description and specific examples are intended for purposes of illustration only and are not intended to limit the scope of the present disclosure.

### DESCRIPTION OF THE DRAWINGS

The drawings described herein are for illustration purposes only and are not intended to limit the scope of the present disclosure in any way.

The forgoing features, objects and advantages of the invention will become apparent to those skilled in the art from the following detailed description of a preferred embodiment, especially when considered in conjunction with the accompanying drawings in which like the numerals in the several views refer to the corresponding parts:

FIG. 1 is a configuration diagram of an adapter module, according to certain embodiments.

FIG. 2 is a block diagram of an adapter module, according to certain embodiments.

FIG. 3 is a schematic diagram of a detailed implementation of an adapter module, according to certain embodiments.

FIG. 4 is a display on a PSG machine receiving multiple input signals from an adapter module, according to certain embodiments.

### DETAILED DESCRIPTION

The following detailed description relates to an adapter module directed toward monitoring patients with sleep disorders in sleep laboratories. The adapter module is more particularly directed at use between a sensor affixed to a patient and a polysomnograph (PSG) machine. The adapter module may be used to receive a signal from a sensor and then convert the signal into multiple signals for display in separate waveforms on a PSG machine. The separate waveforms may include varying levels of detail and may also present waveforms familiar to sleep laboratory practitioners.

The following detailed description includes discussion of sensors affixed to patients, adapter modules, and PSG machines. Additionally, various components of an adapter module are discussed. These include a differential input amplifier, a power supply, and various wave shape filters. These shape filters include an upper airway restriction (UAR) shape filter, an air pressure transducer (APT) filter, and a Thermistor (Thermo) filter.

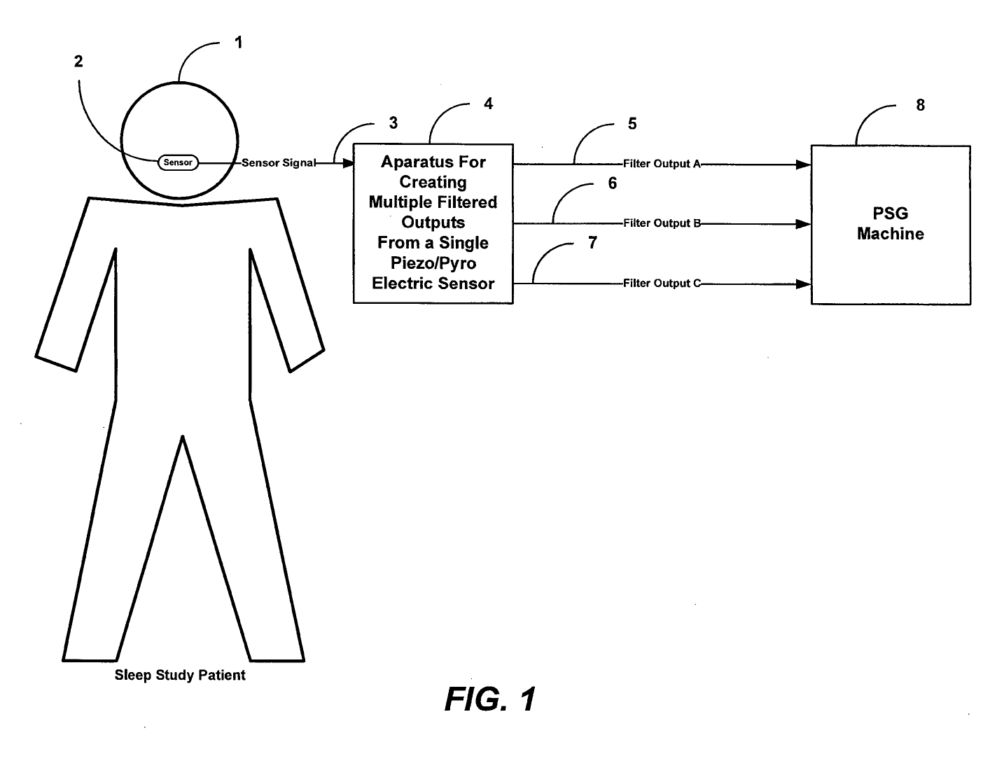

One embodiment of use and configuration of the adapter module is shown with the aid of FIG. 1. A sleep laboratory patient 1 has been outfitted with a sensor 2. A pair of sensor output wire leads 3 connects the sleep laboratory patient 1 to the adapter module 4 for creating multiple filtered outputs from a single sensor. The present embodiment also shows three filtered output wire pairs 5, 6, and 7 connecting the adapter module 4 to a PSG machine 8.

In the present embodiment, the adapter module 4 produces three signals for waveform displays on the PSG machine 8. Each of these signals is transmitted to the PSG machine 8 by an output wire pair. Filter output wire pair 5 transmits a UAR indicating signal showing the most detail on the PSG waveform display. Filter output wire pair 6 transmits an APT type signal showing slightly less detail on the PSG waveform display. Filter output wire pair 7 transmits a Thermo type signal showing minimal detail on the PSG waveform display.

Those skilled in the art will understand and appreciate that various configurations of the apparatuses shown are possible. The adapter module may provide for any number of outputs and inputs. The patient may be fitted with multiple sensors. To the extent necessary to display all of the necessary data, multiple PSG machines could also be used. Additionally, those skilled in the art will understand that various lead configurations are available and that multiple adapter modules could be used.

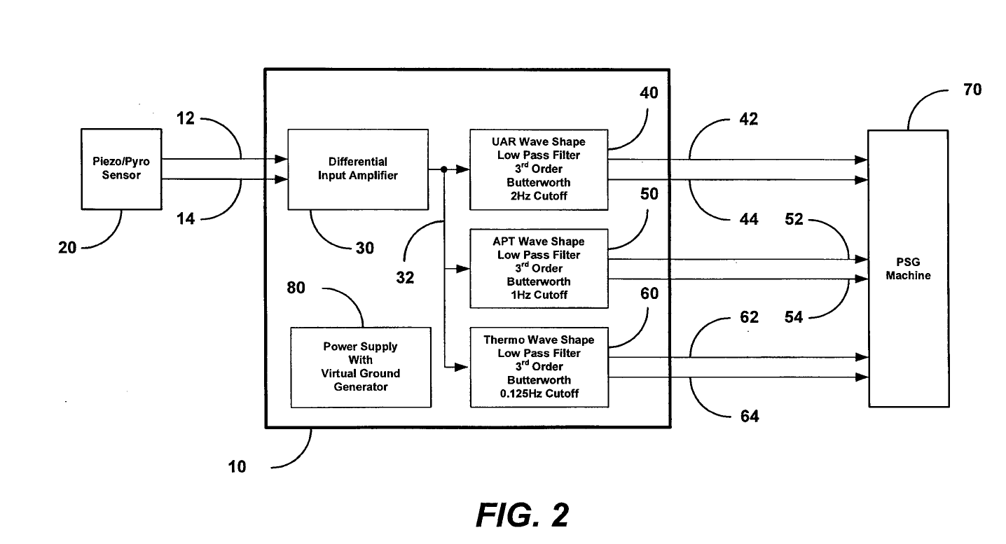

Another embodiment is shown in FIG. 2, which specifically depicts the functional components of an adapter module 10. In this embodiment, a differential input amplifier 30 is shown having a pair of input terminals 12 and 14 to which the leads of a piezo/pyro sensor 20 are connected and an output signal 32. The piezo/pyro sensor 20 is preferably constructed in accordance with the teachings of U.S. Pat. No. 6,491,642, to Stasz and entitled “Pyro/Piezo Sensor,” the teachings of which are hereby incorporated by reference as if fully set forth herein. The sensor 20 is adapted to be placed on a subject's upper lip so that inspiratory and expiratory airflow through the nostrils impinges thereon. Also shown is a power supply 80 and three wave shape filters. The three wave shape filters include a UAR wave shape filter 40, an APT wave shape filter 50, and a Thermo wave shape filter 60. Further included are lines 42, 44, 52, 54, 62, 64, and PSG machine 70.

The differential input amplifier 30 comprises an instrumentation-type amplifier which functions to increase the common-mode rejection of the adapter system to make it less susceptible to 60 Hz noise present in the environment as well as to motion artifacts. Without limitation, the differential input amplifier may have a gain in the range of 2 to 10 with about 6.2 being quite adequate.

The output signal 32 from the differential input amplifier 30 is applied to a bank of three third order Butterworth low pass filters 40, 50, and 60. The inputs of the third order Butterworth filters 40, 50, and 60 are connected to the output terminal of the differential input amplifier 30. Those skilled in the art understand that the literature covering filter responses is vast and that the type of filter response is neither limited to a third order filter nor is it limited to a Butterworth response. Other filter responses may be used such as, but not limited to, Bessel, Elliptic, Chebyshev, BiQuad, State Variable, Infinite Impulse, or Finite Impulse.

In the present embodiment, the cut-off frequency for the UAR wave shaped third order Butterworth low pass filter 40 is 2 Hz creating a PSG display waveform that allows for the indication and diagnosis of UAR's in sleeping patients. Those skilled in the art will understand and appreciate that the cut-off frequency for the UAR filter, while not limited to this range, may vary from 1.5 Hz to 10 Hz.

In the present embodiment, the cut-off frequency for the APT wave shaped third order Butterworth low pass filter 50 is 1 Hz. This creates a PSG display waveform that would have been produced had an APT sensor been used directly with a PSG machine 70 in lieu of the piezo/pyro sensor in conjunction with the adapter module 10. Those skilled in the art will understand and appreciate that the cut-off frequency for the APT filter, while not limited to this range, may vary from 0.5 HZ to 1.5 Hz.

In the present embodiment, the cut-off frequency for the Thermo wave shaped third order Butterworth low pass filter 60 is 0.125 Hz. This creates a PSG display waveform that would have been produced had a Thermo sensor been used directly with a PSG machine 70 in lieu of the piezo/pyro sensor in conjunction with the adapter module 10. Those skilled in the art will understand and appreciate that the cut-off frequency for the Thermo filter, while not limited to this range, may vary from 0.01 HZ to 0.5 Hz.

In the present embodiment, the third order low pass filter 40, or UAR filter, is effective to pass the UAR type signal relating to respiratory activity directly to an input jack of the PSG machine 70 by way of lines 42 and 44 respectively. The third order low pass filter 50, or APT filter, is effective to pass the APT type signal relating to respiratory activity directly to an input jack of the PSG machine 70 by way of lines 52 and 54 respectively. The third order low pass filter 60, or Thermo filter, is effective to pass the Thermo type signal relating to respiratory activity directly to an input jack of the PSG machine 70 by way of lines 62 and 64 respectively.

Those skilled in the art will understand and appreciate that various functional components could be re-arranged and different numbers of these components used. Various types of sensors can be used and the current disclosure is not limited to a piezo/pyro electric sensor. Any number of filters could be used with various frequency cut-offs, which would produce various filter responses. The current disclosure is not limited to producing UAR, APT, and Thermo type signals. The cut-off frequency can be adjusted to provide for a wide range of filter responses and thus a wide range of signals that may be desired by sleep physicians to experiment with other yet unknown and undetermined filter types and responses in order to advance the science of sleep medicine.

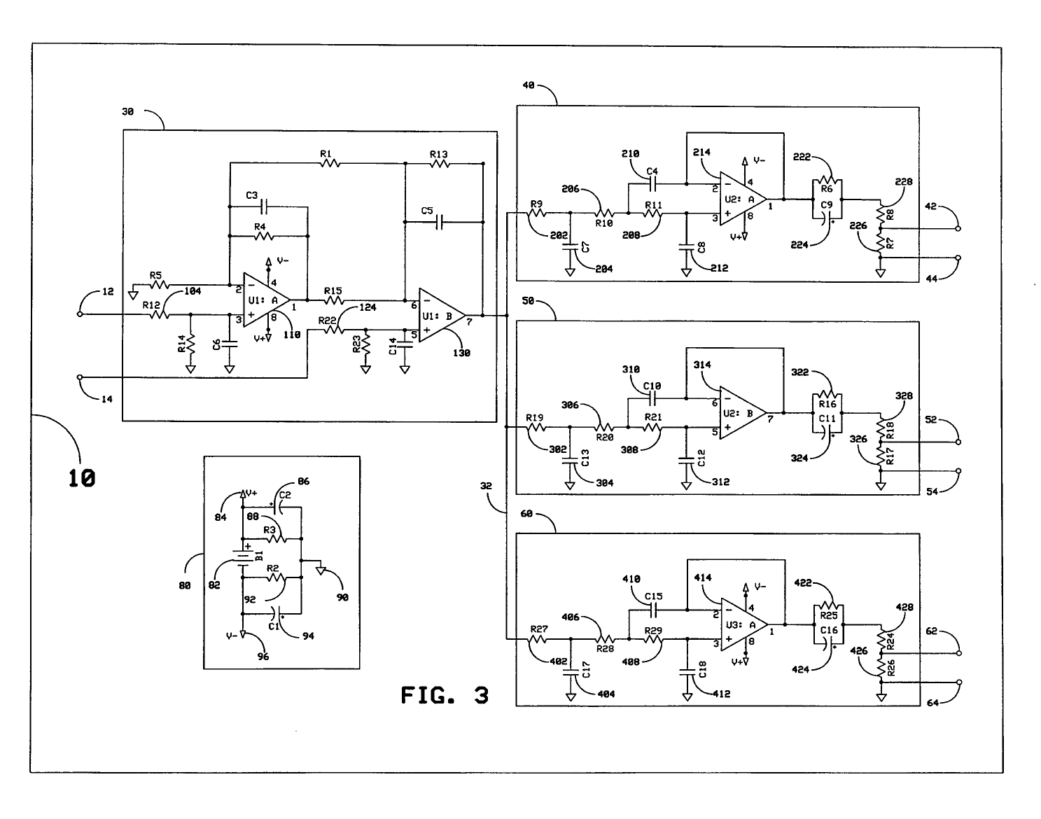

Having described one embodiment of an overall configuration of the adapter module 10 with the aid of FIG. 2, a more detailed explanation of a specific embodiment of the adapter module 10 will now be presented. In that regard, reference is made to the block diagram of FIG. 3, which describes in greater detail, certain embodiments of the building blocks of the adapter module 10.

In one embodiment, the adapter module 10 may be integral with the cable used to couple a piezo/pyro sensor to a PSG machine. In this embodiment, it incorporates its own power supply and virtual ground generator 80. A single lithium battery 82 with a positive battery voltage terminal 84 and a negative battery voltage terminal 96 is included. Also included is a resistor 88 connecting the positive battery voltage terminal to a virtual ground point 90. Further included is a resistor 92 connecting the negative battery voltage terminal to the virtual ground point 90. In the present embodiment, the resistors 88 and 92 are equal in value in the virtual ground point 90 configuration. In this embodiment, a polarized capacitor 86 is included connected in parallel with resistor 88 to form a low alternate current (ac) impedance return path from the positive battery terminal 84 to the virtual ground point 90. A polarized capacitor 94 is also included and is connected in parallel with resistor 92 to form a low alternating (ac) impedance return path from the negative battery terminal 96 to the virtual ground point 90.

Those skilled in the art will understand and appreciate that other arrangements are available for creating a virtual ground. For example, an off-the-shelf integrated circuit could be used such as the TLE2426 Virtual Ground Generator IC available from Texas Instruments. Yet another way of creating a virtual ground is to use a standard operational amplifier in a unity non-inverting gain configuration with the non-inverting input to be the summing node for two equal resistors with their remaining leads tied to the positive voltage terminal 84 and the negative battery voltage terminal 96 respectively.

Referring now to the differential input amplifier 30, in one embodiment, the input terminals 12 and 14 are respectively coupled, via resistors 104 and 124 to the non-inverting inputs of operational amplifiers 110 and 130. Those skilled in the art will appreciate that the operational amplifiers (OpAmps), configured as shown, are typical instrumentation type amplifiers designed to produce a predetermined gain while rejecting common-mode noise. In this embodiment, the output from the differential input amplifier circuit 30 appears at junction 32 and feeds the three third order Butterworth low-pass filter circuits 40, 50, and 60.

Reference is now made to filter circuit 40. In one embodiment, the input appearing at junction 32 is applied, via series connected resistors 202, 206 and 208, to the non-inverting input of an operational amplifier 214. The resistors 202, 206, and 208 along with capacitors 204, 210, and 212 cooperate with the operational amplifier 214 to function as a low-pass filter. The output of the 214 operational amplifier feeds an AC/DC (alternate current/direct current) coupling circuit consisting of a resistor 222 and a capacitor 224. When the adapter module operates with a PSG machine input that requires AC coupled signals only, resistor 222 is not populated in the adapter but ac-coupling capacitor 224 is populated. When the adapter operates with a PSG machine input that requires DC coupled signals, resistor 222 is populated and capacitor 224 is not populated.

The AC/DC coupling circuit, being populated either with resistor 222 or capacitor 224 connects to a voltage divider. The voltage divider includes resistors 226 and 228 and is used to drop the piezo/pyro based signal component to acceptable levels of a PSG machine 80.

The cut-off frequency of the low pass filter circuit 40, or the UAR filter, may be established by setting the values of the resistors 202, 206 and 208 and the capacitors 204, 210 and 212. As discussed, in one embodiment, this cut-off frequency may be set to about 2 Hz.

Reference is now made to filter circuit 50. In one embodiment, the input appearing at junction 32 is applied, via series connected resistors 302, 306 and 308, to the non-inverting input of an operational amplifier 314. The resistors 302, 306, and 308, along with capacitors 304, 310 and 312 cooperate with the operational amplifier 314 to function as a low-pass filter. The output of the 314 operational amplifier feeds an AC/DC (alternate current/direct current) coupling circuit consisting of a resistor 322 and a capacitor 324. When the adapter operates with a PSG machine input that requires AC coupled signals only, resistor 322 is not populated in the adapter but ac-coupling capacitor 324 is populated. When the adapter operates with a PSG machine input that requires DC coupled signals, resistor 322 is populated and capacitor 324 is not populated.

The AC/DC coupling circuit, being populated either with resistor 322 or capacitor 324 connects to a voltage divider. The voltage divider includes resistors 326 and 328 and is used to drop the piezo/pyro based signal component to acceptable levels of a PSG machine 80.

The cut-off frequency of the low pass filter circuit 50, or the APT filter, may be established by setting the values of the resistors 302, 306 and 308 and the capacitors 304, 310 and 312. As discussed, in one embodiment, this cut-off frequency may be set to about 1 Hz.

Reference is now made to filter circuit 60. In one embodiment, the input appearing at junction 32 is applied, via series connected resistors 402, 406 and 408, to the non-inverting input of an operational amplifier 414. The resistors 402, 406, and 408, along with capacitors 404, 410 and 412 cooperate with the operational amplifier 414 to function as a low-pass filter. The output of the 414 operational amplifier feeds an AC/DC (alternate current/direct current) coupling circuit consisting of a resistor 422 and a capacitor 424. When the adapter operates with a PSG machine input that requires AC coupled signals only, resistor 422 is not populated in the adapter but ac-coupling capacitor 424 is populated. When the adapter operates with a PSG machine input that requires DC coupled signals, resistor 422 is populated and capacitor 424 is not populated.

The AC/DC coupling circuit, being populated either with resistor 422 or capacitor 424 connects to a voltage divider. The voltage divider includes resistors 426 and 428 and is used to drop the piezo/pyro based signal component to acceptable levels of a PSG machine 80.

The cut-off frequency of the low pass filter circuit 60, or the Thermo filter, may be established by setting the values of the resistors 402, 406 and 408 and the capacitors 404, 410 and 412. As discussed, in one embodiment, this cut-off frequency may be set to about 0.125 Hz.

The list of specific components used to assemble a printed circuit board assembly is known in the industry as a Bill-of-Materials (BOM). Below is an example of a BOM for one embodiment of the components of FIG. 3.
B1 BR2330A/FA
R6 100
R16 100
R25 100
C8 0.056 uF
C12 0.056 uF
C18 0.056 uF
C3 0.1 uF
C5 0.1 uF
C7 0.39 uF
C13 0.39 uF
C17 0.39 uF
R4 100 k
R5 100 k
R13 100 k
R15 100 k
C6 100 pF
C14 100 pF
C1 10 uF
C2 10 uF
R12 1 k
R22 1 k
C4 1 uF
C10 1 uF
C15 1 uF
R8 24.3 k
R18 24.3 k
R24 24.3 k
R9 270 k
R10 270 k
R11 270 k
R27 3.3 M
R28 3.3 M
R29 3.3 M
R2 330 k
R3 330 k
R1 47.5 k
C9 47 uF
C11 47 uF
C16 47 uF
R14 5.1 M
R23 5.1 M
R19 560 k
R20 560 k
R21 560 k
R7 6.8 M
R17 6.8 M
R26 6.8 M
U1: A LMC6442AIM
U1: B LMC6442AIM
U2: A LMC6442AIM
U2: B LMC6442AIM
U3: A LMC6442AIM

Those of skill in the art will understand and appreciate that this BOM is simply exemplary and a wide array of values and a wide array of combinations of the above elements can be used.

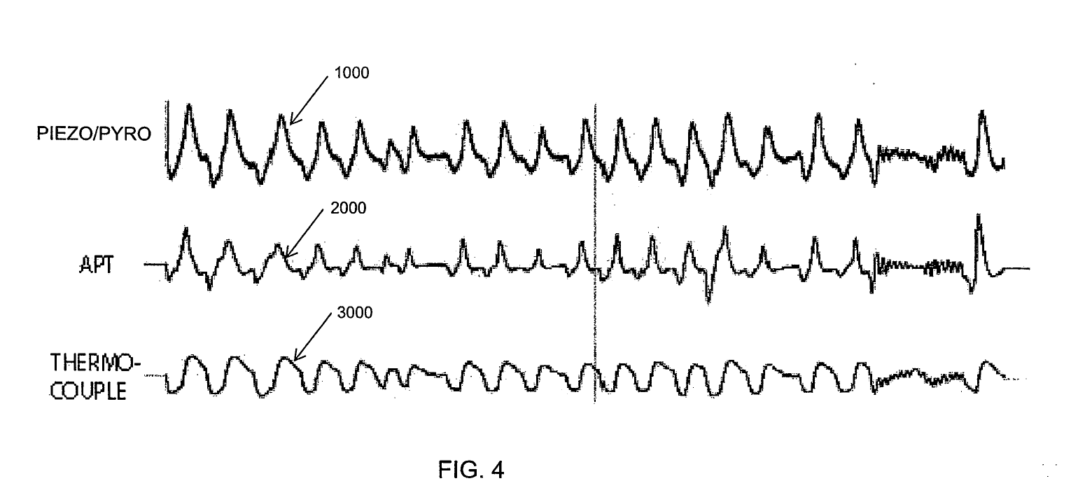

Referring now to FIG. 4, in one embodiment, three signals received by a PSG machine are simultaneously displayed in waveform on a PSG screen. In the present embodiment, a waveform 1000 produced from a UAR filter is shown. Also shown is a waveform 2000 produced from an APT filter and a waveform 3000 produced from a Thermo filter. In the present embodiment the input signals from the various filters provide varying levels of detail. Waveform 1000 from the UAR filter provides the most detail and includes detailed UAR information. Waveform 2000 from the APT filter shows slightly less detail and waveform 3000 shows minimal detail. In the present embodiment, waveforms 2000 and 3000 are more likely to be familiar waveforms to sleep disorder practitioners and waveform 1000 is less likely to be a familiar waveform.

Those of skill in the art will understand and appreciate that any number of waveforms could be produced using a larger number of filters in the adapter module. Moreover, the waveforms produced can vary and are not limited to UAR, APT, and Thermo type waveforms.

During operation, in one embodiment, a sleep laboratory patient may be fitted with a piezo/pyro electric film sensor that includes a circuit similar to that described in detail here. The circuit may then be further connected to a PSG machine. As the patient breathes and/or sleeps, sleep scientists, sleep physicians, and sleep technicians may then be able to see, detect and properly diagnose specific sleep disorders and diseases. These disorders may include abnormal respiratory events. Moreover, the present embodiment provides the ability to review familiar waveforms which may signify familiar sleep disorders, but also provides the ability to review more detailed information regarding UAR's at the same time. Thus, the present embodiment may allow practitioners to more thoroughly understand the disorders of patients and provide better care.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

The description of the various embodiments is merely exemplary in nature and, thus, variations that do not depart from the gist of the examples and detailed description herein are intended to be within the scope of the present disclosure. Such variations are not to be regarded as a departure from the spirit and scope of the present disclosure.

What is claimed is:

1. An apparatus for creating multiple filtered outputs for a polysomnograph (PSG) machine from a single sensor input, comprising:
an electronic signal processing circuit configured to receive a single sensor input and to produce, using the single sensor input, multiple different output signals, the multiple different output signals including:
a first output indicative of an upper airway restriction (UAR);
a second output indicative of an airway pressure during respiration; and
a third output indicative of an airway air temperature during respiration.

2. The apparatus of claim 1, wherein the second output is indicative of a difference in the airway pressure during respiration, and the third output is indicative of a difference in the airway air temperature during respiration.

3. The apparatus of claim 1, wherein the electronic signal processing circuit is configured to receive the single sensor input from a piezo/pyro sensor, sized and shaped to be affixed to a subject's upper lip and configured to receive respiration information from the subject.

4. The apparatus of claim 1, wherein the electronic signal processing circuit is configured to provide information about at least one of the produced multiple different output signals to a user.

5. The apparatus of claim 1, wherein the electronic signal processing circuit is configured to produce the multiple different output signals for a polysomnograph (PSG) machine from the single sensor input.

6. The apparatus of claim 1, wherein the electronic signal processing circuit includes a differential amplifier configured to amplify the single sensor input and to attenuate common-mode noise.

7. The apparatus of claim 1, wherein the electronic signal processing circuit includes a UAR shape filter configured to produce the first output, an air pressure transducer (APT) shape filter configured to produce the second output, and a thermistor (Thermo) shape filter configured to produce the third output.

8. The apparatus of claim 7, wherein the UAR shape filter includes a first low-pass filter having a cut-off frequency between 1.5 Hz and 10 Hz, the APT shape filter includes a second low-pass filter having a cut-off frequency between 0.5 Hz and 1.5 Hz, and the Thermo shape filter includes a third low-pass filter having a cut-off frequency between 0.01 Hz and 0.5 Hz.

9. The apparatus of claim 1, wherein the electronic signal processing circuit is configured to produce the second output to resemble an air pressure transducer (APT) waveform on a polysomnograph (PSG) machine, and to produce the third output to resemble a thermistor (Thermo) waveform on the PSG machine.

10. The apparatus of claim 1, wherein the electronic signal processing circuit is configured to be integrated into a cable coupling a piezo/pyro sensor to a polysomnograph (PSG) machine.

11. A system for creating multiple filtered outputs for a polysomnograph (PSG) machine from a single sensor input, comprising:
a piezo/pyro sensor, sized and shaped to be affixed to a subject's upper lip, the piezo/pyro sensor configured to receive respiration information from the subject;
an electronic signal processing circuit configured to receive information from the piezo/pyro sensor and to produce, using the piezo/pyro sensor input, multiple different output signals, the multiple different output signals including:
a first output indicative of an upper airway restriction (UAR);
a second output indicative of an airway pressure during respiration; and
a third output indicative of an airway air temperature during respiration; and
a polysomnograph (PSG) machine configured to receive the multiple different output signals from the electronic signal processing circuit and to display information about at least one of the received first output, the received second output, or the received third output to a user.

12. The system of claim 11, including a cable configured to couple the piezo/pyro sensor to the PSG machine, wherein the electronic signal processing circuit is configured to be integrated into the cable.

13. A method for creating multiple filtered outputs for a polysomnograph (PSG) machine from a single sensor input, comprising:
receiving a single sensor input;
producing, using the single sensor input, multiple different output signals, the producing including:
producing a first output indicative of an upper airway restriction (UAR);
producing a second output indicative of an airway pressure during respiration; and
producing a third output indicative of an airway air temperature during respiration.

14. The method of claim 13, wherein the receiving the single sensor input includes receiving a single sensor input from a piezo/pyro sensor, sized and shaped to be affixed to a subject's upper lip and configured to receive respiration information from the subject.

15. The method of claim 14, wherein the producing the multiple different output signals includes using an electronic signal processing circuit integrated into a cable coupling the piezo/pyro sensor to a polysomnograph (PSG) machine.

16. The method of claim 13, including providing information about at least one of the produced multiple different output signals to a user.

17. The method of claim 13, including receiving the produced multiple different output signals using a polysomnograph (PSG) machine and providing information about at least one of the received multiple different output signals to a user.

18. The method of claim 13, wherein the producing the first output includes using a first UAR shape filter, the producing the second output includes using an air pressure transducer (APT) shape filter, and the producing the third output includes using a thermistor (Thermo) shape filter.

19. The method of claim 18, wherein the using the UAR shape filter includes using a first low-pass filter having a cut-off frequency between 1.5 Hz and 10 Hz, the using the APT shape filter includes using a second low-pass filter having a cut-off frequency between 0.5 Hz and 1.5 Hz, and the using the Thermo shape filter includes using a third low-pass filter having a cut-off frequency between 0.01 Hz and 0.5 Hz.

20. The method of claim 13, wherein the producing the second output includes producing output to resemble an air pressure transducer (APT) waveform on a polysomnograph (PSG) machine, and the producing the third output includes producing output to resemble a thermistor (Thermo) waveform on the PSG machine.
